(19) United States
(12) Patent Application Publication
(10) Pub. No.: US 2019/0285639 A1
(43) Pub. Date: Sep. 19, 2019

(54) SYSTEMS AND METHODS FOR OPTICAL SENSING OF BIOMOLECULAR TARGETS

(71) Applicant: Integrated Nano-Technologies, Inc., Henrietta, NY (US)

(72) Inventors: Dennis M. Connolly, Rochester, NY (US); Richard S. Murante, Rochester, NY (US); Nathaniel E. Wescott, West Henrietta, NY (US)

(73) Assignee: Integrated Nano-Technologies, Inc., Henrietta, NY (US)

(21) Appl. No.: 16/345,175

(22) PCT Filed: Oct. 26, 2017
(86) PCT No.: PCT/US17/58559

§ 371 Date: Apr. 25, 2019

Related U.S. Application Data
(60) Provisional application No. 62/413,144, filed on Oct. 26, 2016.

(51) Int. Cl. G01N 33/58; B01L 3/00; G01N 33/543; G01N 15/14

(52) U.S. Cl. CPC G01N 33/587; G01N 2015/1006; G01N 33/54373; G01N 15/1434; G01N 15/1463; B01L 2200/0668; B01L 2300/0816; B01L 2300/0819; B01L 2300/0887; B01L 2300/16; B01L 2400/043; B01L 2400/0478; B01L 2400/0622; B01L 2400/0644; B01L 3/502761

(57) ABSTRACT

A system for detection of a target molecule includes an imager, a flow cell having a functionalized surface, a light source, and a magnet. The functionalized surface is configured to bind a target molecule attracted to the functionalized surface via the magnet. The target molecule is configured to bind a nanoparticle and the light source is configured to direct a light beam toward the bound nanoparticle. Light from the nanoparticle is captured by the imager and analyzed to detect the presence of the target molecule.

### CROSS-REFERENCE TO RELATED APPLICATIONS

This application claims the benefit of and priority to U.S. Provisional Patent Application Ser. No. 62/413,144, filed Oct. 26, 2016 and entitled “AUTOMATED NUCLEIC ACID DETECTION AND QUANTITATION WITH OPTICAL SENSING,” the entirety of which is incorporated herein by reference.

### BACKGROUND

The subject matter disclosed herein relates to a detecting target molecules, such as nucleic acid molecules and, more particularly, to systems for optical sensing of the target molecules.

Various methods have developed for analyzing biological samples and detecting the presence of target molecules, such as nucleic acid molecules. These methods can be used, for example, in detecting pathogens in samples.

Typically, detection methods use disruption techniques, such as Polymerase Chain Reaction (PCR) to extract and replicate nucleic acid molecules from a sample. PCR is a technique that allows for replicating and amplifying trace amounts of DNA fragments into quantities that are sufficient for analysis. As such, PCR can be used in a variety of applications, such as DNA sequencing and detecting DNA fragments in samples.

An electronic sensor for detection of specific target nucleic acid molecules can include capture probes immobilized on a sensor surface between a set of paired electrodes. An example of a system and method for detecting target nucleic acid molecules is described in U.S. Pat. No. 7,645,574, the entirety of which is herein incorporated by reference. Following PCR, amplified products or amplicons derived from targeted pathogen sequences are captured by the probes. Nano-gold clusters, functionalized with a complementary sequence, are used for localized hybridization to the amplicons. Subsequently, using a short treatment with a gold developer reagent, the nano-gold clusters serve as catalytic nucleation sites for metallization, which cascades into the development of a fully conductive film. The presence of the gold film shorts the gap between the electrodes and is measured by a drop in resistance, allowing the presence of the captured amplification products to be measured. However, such sensors can be insensitive to small quantities of target molecules, resulting in false negative results or a failure to detect the target molecules.

### SUMMARY

A system for detection of a target molecule includes an imager, a flow cell having a functionalized surface, a light source, and a magnet. The functionalized surface is configured to bind a target molecule attracted to the functionalized surface via the magnet. The target molecule is configured to bind a nanoparticle and the light source is configured to direct a light beam toward the bound nanoparticle. Light from the target molecule or nanoparticle is captured by the imager and analyzed to detect the presence of the target molecule.

In an embodiment, a system for detecting target molecules in a sample is described. The system includes an imager and a flow cell having a transparent surface and a functionalized surface. The functionalized surface includes a plurality of capture probes configured to bind the target molecules. A magnet is positioned opposite the functionalized surface and is configured to direct the target molecules to the functionalized surface. A light source is configured to direct a light beam at bound target molecules. The imager is configured to capture light from the target molecules to detect the presence of the target molecules.

In another embodiment, a method for detecting a target molecule in a sample with a sensor is described. The sensor includes an imager, a flow cell, a magnet, and a light source. The flow cell includes a functionalized surface having a plurality of capture probes coupled to the functionalized surface. The method includes binding the target molecule to a magnetic particle and directing the magnetic particle and target molecule to the functionalized surface via the magnet. The method further includes binding the target molecule to one of the plurality of capture probes and binding a nanoparticle to the target molecule. A light beam from the light source is directed at the nanoparticle and light from the nanoparticle is captured at the imager. The light from the nanoparticle is analyzed to detect the target molecule.

In yet another embodiment, a method for detecting a target molecule in a sample with a sensor is described. The sensor includes an imager, a flow cell, a magnet, and a light source. The flow cell includes a functionalized surface having a plurality of capture probes coupled to the functionalized surface. The method includes binding the target molecule to a magnetic particle and directing the magnetic particle and target molecule to the functionalized surface via the magnet. The method further includes binding the target molecule to one of the plurality of capture probes. A light beam from the light source is directed at the magnetic particle and light from the magnetic particle is captured at the imager. The light from the from the magnetic particle is analyzed to detect the target molecule.

An advantage that may be realized in the practice of some disclosed embodiments is increased sensitivity of nucleic acid sensors and improved detection of low concentrations of target materials.

The above embodiments are exemplary only. Other embodiments are within the scope of the disclosed subject matter.

### BRIEF DESCRIPTION OF THE DRAWINGS

So that the manner in which the features of the invention can be understood, a detailed description of the invention may be had by reference to certain embodiment, some of which are illustrated in the accompanying drawings. It is to be noted, however, that the drawings illustrate only certain embodiments of this invention and are therefore not to be considered limiting of its scope, for the scope of the disclosed subject matter encompasses other embodiments as well. The drawings are not necessarily to scale, emphasis generally being placed upon illustrating the features of certain embodiments of the invention. In the drawings, like numerals are used to indicate like parts throughout the various views.

FIG. 1 is a perspective view of a portable diagnostic assay system operative to accept one of a plurality of disposable cartridges configured to test fluid samples of collected blood/food/biological samples;

FIG. 2 is an exploded perspective view of one of the disposable cartridges configured to test a blood/food/biological sample;

FIG. 3 is a top view of the one of the disposable cartridges illustrating a variety of assay chambers including a central assay chamber, one of which contains an assay chemical suitable to breakdown the fluid sample to detect a particular attribute of the tested fluid sample;

FIG. 4 is a bottom view of the disposable cartridge shown in FIG. 3 illustrating a variety of channels operative to move at least a portion of the fluid sample from one chamber to another for the purpose of performing multiple operations on the fluid sample.

FIG. 5 is a diagram of an embodiment of a sensor system having a functionalized surface;

FIG. 6 is a flowchart illustrating an embodiment of a method of detecting a target molecule;

FIG. 7 is a diagram of the sensor system of FIG. 5 with target molecules bound to magnetic particles;

FIG. 8A is cross-sectional illustration of an embodiment of a magnetic particle;

FIG. 8B is a cross-sectional illustration of another embodiment of a magnetic particle;

FIG. 8C is an illustration of an embodiment of a magnetic particle bound with nanoparticles;

FIG. 8D is an illustration of another embodiment of a magnetic particle bound with nanoparticles;

FIG. 8E is an illustration of an embodiment of a target molecule bound with a magnetic particle and a nanoparticle;

FIG. 8F is an illustration of another embodiment of a target molecule bound with a magnetic particle and nanoparticles;

FIG. 8G is an illustration of yet another embodiment of a target molecule bound with a nanoparticle and magnetic particles;

FIG. 9 is a diagram of the sensor system of FIGS. 5 and 7 with the target molecules bound to the functionalized surface;

FIG. 10 is a diagram of the sensor system of FIGS. 5 and 7-8 with functionalized nanoparticles bound to the target molecules;

FIG. 11 is a diagram of the sensor system of FIGS. 5, 7-8, and 10 with a light source directed at the functionalized nanoparticles;

FIG. 12A is an embodiment of scattering signatures of 50 nm monodispersed nanoparticles under dark field microscopy;

FIG. 12B is an embodiment of scattering signatures of 100 nm monodispersed nanoparticles under dark field microscopy;

FIG. 13 is a comparison of scattering signatures of developed nanoparticles versus undeveloped nanoparticles under dark field microscopy;

FIG. 14 is an illustration of an embodiment of an optical sensor system;

FIG. 15A is an enlarged partial illustration of the optical sensor system of FIG. 14 with the magnet retracted;

FIG. 15B is an enlarged partial illustration of the optical sensor system of FIG. 14 with the magnet extended;

FIG. 16 is a side view illustration of an embodiment of an optical instrument incorporating the optical sensor system of FIG. 14;

FIG. 17A is a diagram of another embodiment of a sensor system having a functionalized surface;

FIG. 17B is a diagram of the sensor system of FIG. 17A with target molecules bound to the functionalized surface; and

FIG. 17C is a diagram of the sensor system of FIGS. 17A-17B having nanoparticles bound to the target molecules.

Corresponding reference characters indicate corresponding parts throughout several views. The examples set out herein illustrate several embodiments, but should not be construed as limiting in scope in any manner.

### DETAILED DESCRIPTION

A disposable cartridge is described for use in a portable/automated assay system such as that described in commonly-owned, co-pending U.S. patent application Ser. No. 15/157,584 filed May 18, 2016 entitled “Method and System for Sample Preparation” which is hereby included by reference in its entirety. While the principal utility for the disposable cartridge includes DNA testing, the disposable cartridge may be used to detect any of a variety of diseases which may be found in either a blood, food or biological detecting hepatitis, autoimmune deficiency syndrome (AIDS/HIV), diabetes, leukemia, graves, lupus, multiple myeloma, etc., just naming a small fraction of the various blood borne diseases that the portable/automated assay system may be configured to detect. Food diagnostic cartridges may be used to detect Salmonella, E-coli, Staphylococcus aureus or dysentery. Diagnostic cartridges may also be used to test samples from insects and specimen. For example, blood diagnostic cartridges may be dedicated cartridges useful for animals to detect diseases such as malaria, encephalitis and the west nile virus, to name but a few.

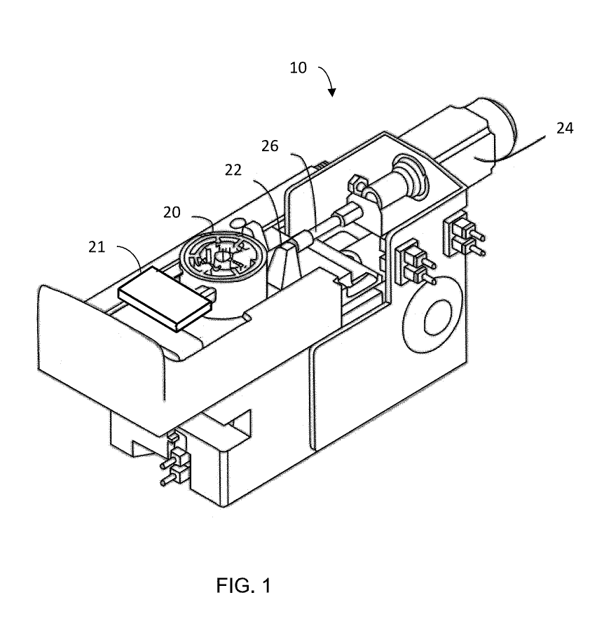

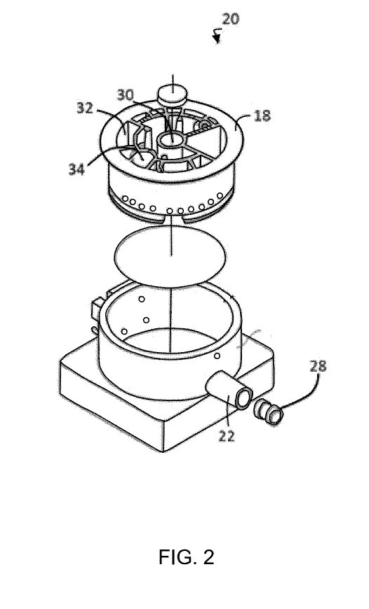

More specifically, and referring to FIGS. 1 and 2, a portable assay system 10 receives any one of a variety of disposable assay cartridges 20, each selectively configured for detecting a particular attribute of a fluid sample, each attribute potentially providing a marker for a blood, food or biological (animal borne) disease. The portable assay system 10 includes one or more linear and rotary actuators operative to move fluids into, and out of, various compartments or chambers of the disposable assay cartridge 20 for the purpose of identifying or detecting a fluid attribute. More specifically, the cartridge 20 includes a flow cell 21 extending horizontally therefrom. A rotary actuator (not shown) of the portable assay system 10 aligns one of a variety of ports 18P, disposed about a cylindrical rotor 18, with a syringe barrel 22B of a stationary cartridge body 22. The linear actuator 24 displaces a plunger shaft 26 so as to develop pressure i.e., positive or negative (vacuum) in the syringe barrel 22. That is, the plunger shaft 26 displaces an elastomer plunger 28 within the syringe 22 to move and or admix fluids contained in one or more of the chambers 30, 32.

The disposable cartridge 20 provides an automated process for preparing the fluid sample for analysis and/or performing the fluid sample analysis. The sample preparation process allows for disruption of cells, sizing of DNA and RNA, and concentration/clean-up of the material for analysis. More specifically, the sample preparation process of the instant disclosure prepares fragments of DNA and RNA in a size range of between about 100 and 10,000 base pairs. The chambers can be used to deliver the reagents necessary for end-repair and kinase treatment. Enzymes may be stored dry and rehydrated in the disposable cartridge 20, or added to the disposable cartridge 20, just prior to use. The implementation of a rotary actuator allows for a single plunger 26, 28 to draw and dispense fluid samples without the need for a complex system of valves to open and close at various times. This greatly reduces potential for leaks and failure of the device compared to conventional systems. Finally, it will also be appreciated that the system greatly diminishes the potential for human error.

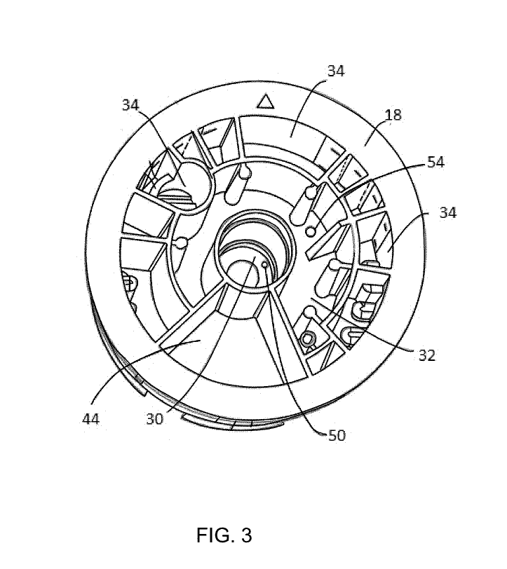

In FIGS. 3 and 4, the cylindrical rotor 18 includes a central chamber 30 and a plurality of assay chambers 32, 34 surrounded, and separated by, one or more radial or circumferential walls. In the described embodiment, the central chamber 30 receives the fluid sample while the surrounding chambers 32, 34 contain a premeasured assay chemical or reagent for the purpose of detecting an attribute of the fluid sample. The chemical or reagents may be initially dry and rehydrated immediately prior to conducting a test. Some of the chambers 32, 34 may be open to allow the introduction of an assay chemical while an assay procedure is underway or in-process. The chambers 30, 32, 34 are disposed in fluid communication, i.e., from one of the ports 18P to one of the chambers 30, 32, 34, by channels 40, 42 molded along a bottom panel 44, i.e., along underside surface of the rotor 18. For example, a first port 18P, corresponding to aperture 42, may be in fluid communication with the central chamber 30, via aperture 50.

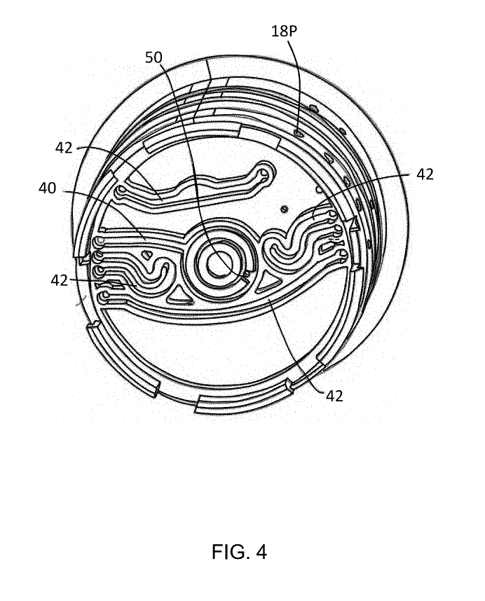

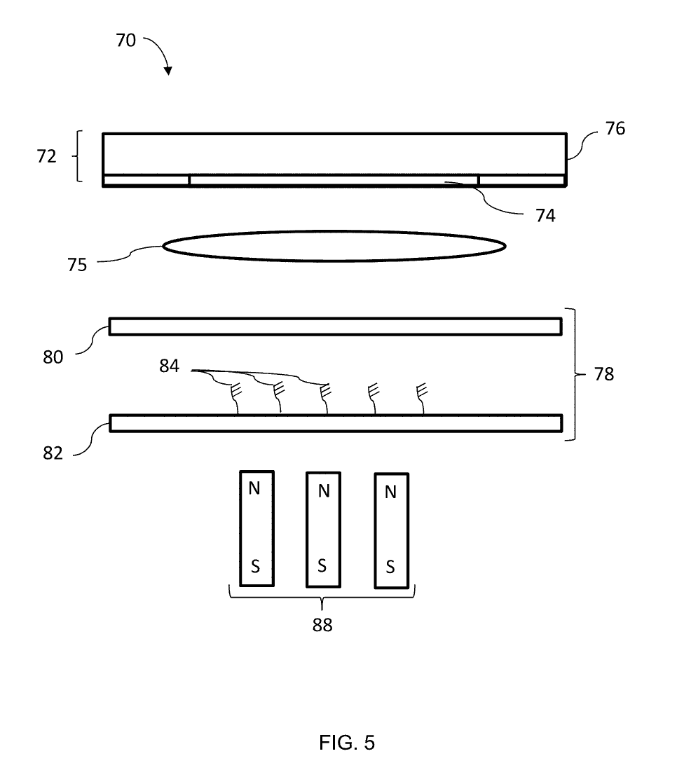

FIG. 5 illustrates an embodiment of a sensor system 70. The sensor system 70 includes an imager 72 configured to capture still images, video, or a combination thereof. For example, the imager 72 can be configured to capture high resolution still images. In the illustrated embodiment, the imager 72 includes a pixel array 74 and array circuitry 76. The pixel array 74 can include any suitable number of pixels. For example, the pixel array 74 can be a high density array including at least six (6) megapixels. In a further example, the camera can have a large field of view. The pixel array 74 is a light sensitive pixel array, such as an active array, passive array, planar Fourier capture array, angle sensitive array, photodiode array, a charge coupled device, a complementary metal-oxide semiconductor (CMOS), or a charge injection device.

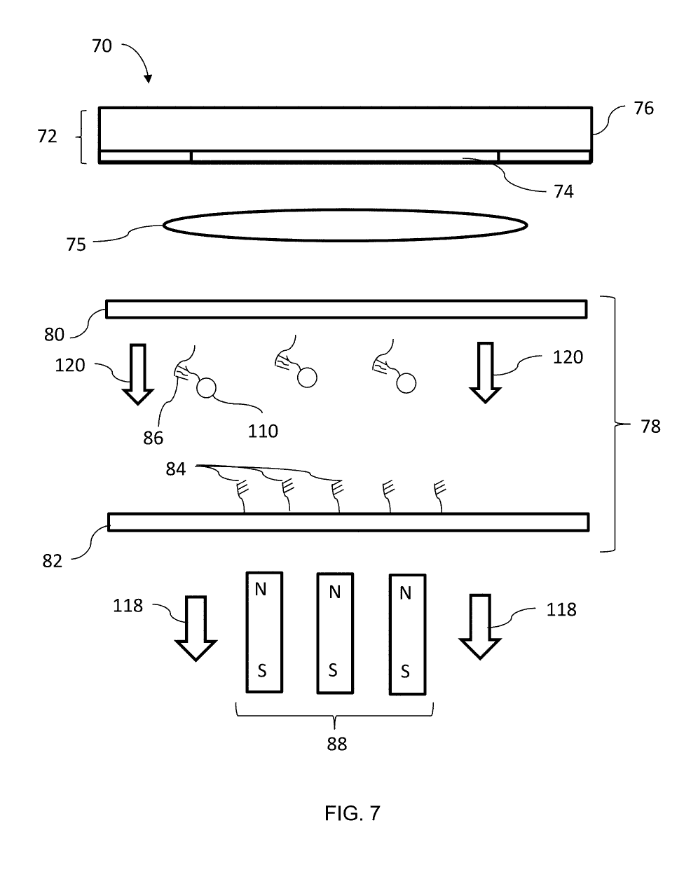

The sensor system 70 also includes a flow cell 78. The flow cell 78 can be formed of any suitable material, such as a polypropylene or polystyrene polymer or glass, among others. In an embodiment, the flow cell is formed by injection molding. The flow cell 78 includes a transparent or optically clear surface 80 and a transparent functionalized surface 82. The functionalized surface 82 includes a plurality of capture probes 84 in the form of a functionalized oxide surface allowing attachment and immobilization of capture probe molecules 84 on the surface 82. The capture probes 84 are designed to capture or bind target molecules 86 (FIG. 7) by interaction between complementary sequences. The target molecules 86 can be collected from a biological sample. The biological sample could be any suitable type of materials, such as blood, mucous, and skin, among others. For example, the target molecules 86 can be protein ligands or DNA segments.

An objective or lens 75 can optionally be positioned between the imager 72 and the flow cell 78. A magnet 88 can be positioned opposite the functionalized surface 82. The magnet 88 can be a single magnet or an array of magnets.

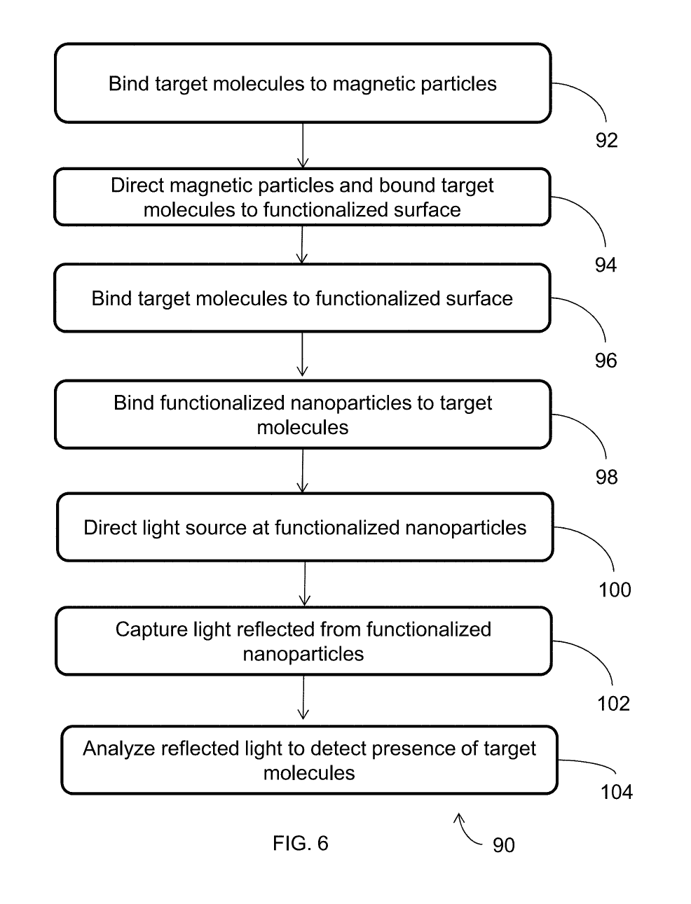

FIG. 6 illustrates an embodiment of a method 90 for detection of a target molecule. The method 90 can be employed by a sensor system, such as the sensor system 70. At block 92, target molecules 86 are bound to magnetic particles 110, as illustrated in FIG. 7. In an embodiment, the target molecules 86 are bound to the magnetic particles 110 before being introduced to the flow cell 78. In another embodiment, the target molecules 86 and magnetic particles 110 are introduced to the flow cell 78 in an unbound state and the target molecules 86 bind to the magnetic particles 110 within the flow cell 78.

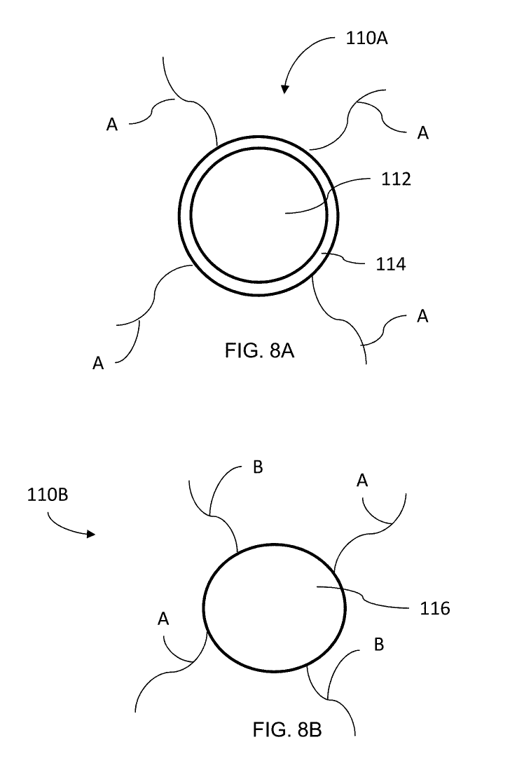

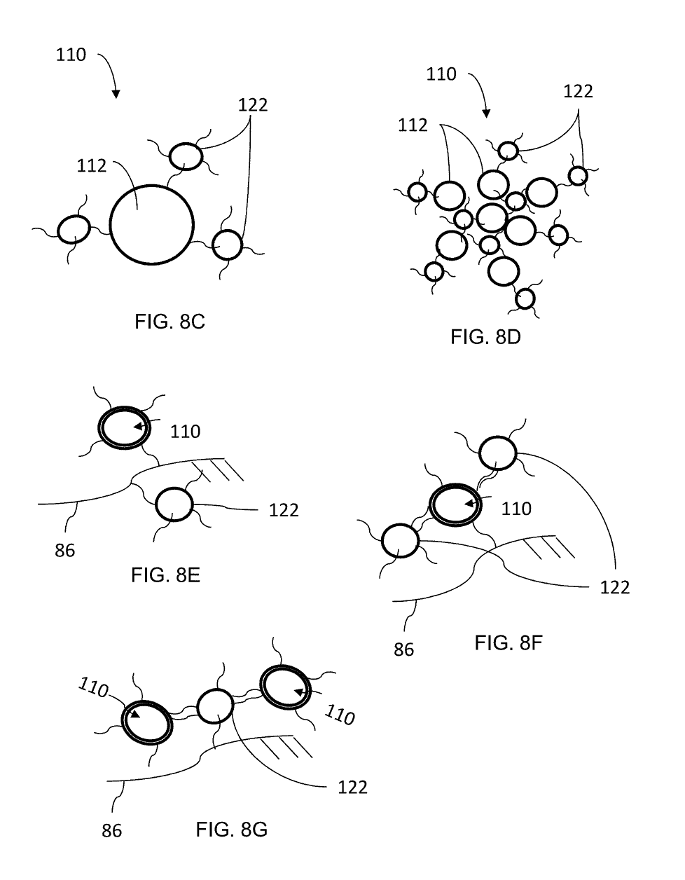

FIGS. 8A-8B illustrate two embodiments of magnetic particles 110. As illustrated in FIG. 8A, in one embodiment the magnetic particle 110A is a composite particle that has a magnetic core 112, formed of a magnetic material such as iron, and a nanoparticle coating 114. For example, the coating 114 can be a gold coating. The coating 114 can be configured to act as a nucleation site for further nanoparticle development. The magnetic particle 110A includes at least one binding site for a ligand A for binding to the target molecules 86. A chemical reactive group such as a thiol, amine, or aldehyde, can mediate or facilitate ligand binding.

As illustrated in FIG. 8B, in another embodiment, the magnetic particle 110B can have a magnetic body 116 formed of a magnetic material, such as iron. The magnetic particle 110B includes at least one binding site or ligand A for binding to a target molecule. In the illustrated embodiment, the magnetic particle 110B further includes at least one binding site or ligand B for binding a magnetic nanoparticle. It is to be understood that the magnetic particle can include any suitable combination of binding sites A, B. For example, the magnetic particle can include both types of binding sites A, B or the magnetic particle can include only target molecule binding sites A.

As illustrated in FIG. 8C, rather than a nanoparticle coating over a magnetic core, the magnetic particle 110 can include a magnetic body 112 and a plurality of nanoparticles 122 bound to the magnetic body 112. Alternatively, as illustrated by FIG. 8D, the magnetic particle 110 can be an alloy, such as a heterogeneous alloy, including a plurality of magnetic bodies 112 bound with a plurality of nanoparticles 122.

As illustrated in FIGS. 8E-8G, the target molecule 86, magnetic particle 110, and nanoparticle 122 can be bound in a variety of arrangements. As illustrated in FIG. 8E, the magnetic particle 110 and nanoparticle 122 can each be bound directly to the target molecule 86. Alternatively and as illustrated in FIG. 8F, the magnetic particle 110 can be bound directly to the target molecule 86 and one or more nanoparticles 122 can be bound to the magnetic particle 110. Alternatively and as illustrated in FIG. 8G, a nanoparticle 122 can be bound directly to the target molecule 86 and one or more magnetic particles 110 can be bound to the nanoparticle 122.

Returning to FIG. 6, at block 94, the bound magnetic particles 110 and target molecules 86 are directed or moved to the functionalized surface 82. Referring to FIG. 7, the magnet 88 is coupled to an actuator (not shown) configured to move the magnet 88 toward (retracted) and away from (extended) the functionalized surface 82. As the magnet 88 is moved away 118 from the functionalized surface 82, the magnetic particles 110, attracted to the magnet 88, move 120 toward the functionalized surface 82. As the target molecules 86 are bound to the magnetic particles 110, the target molecules 86 are directed or drawn by the magnetic particles 110 toward the functionalized surface 82.

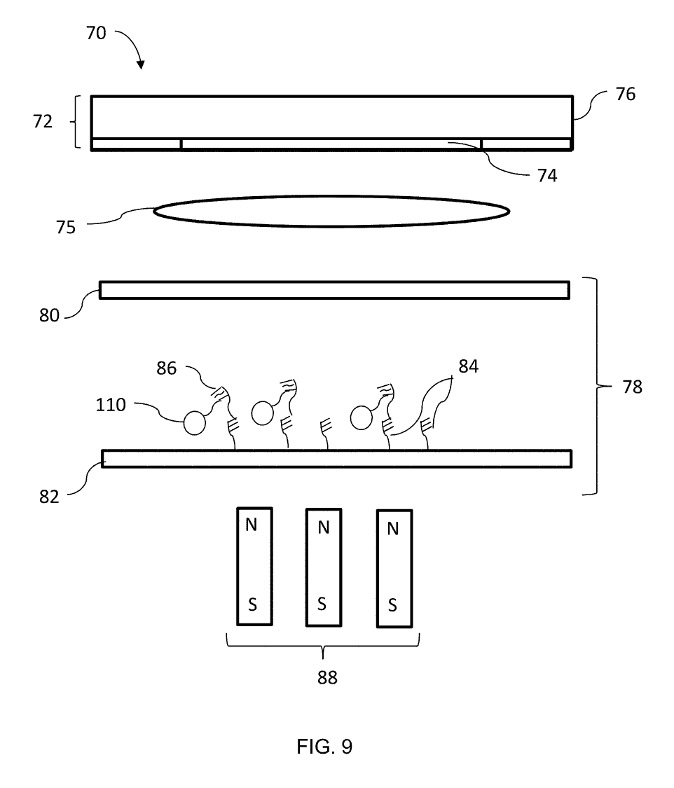

Returning to FIG. 6, at block 96, the target molecules 86 are bound to the capture probes 84 of the functionalized surface 82 as illustrated in FIG. 9. In the illustrated embodiment, the magnetic particles 110 remain bound to the bound target molecules 86. Alternatively, the target molecules 86 can be denatured to unbind the magnetic particles 110 from the target molecules 86 when the target molecules 86 reach the functionalized surface 82, following which the target molecules 86 can bind to the functionalized surface 82.

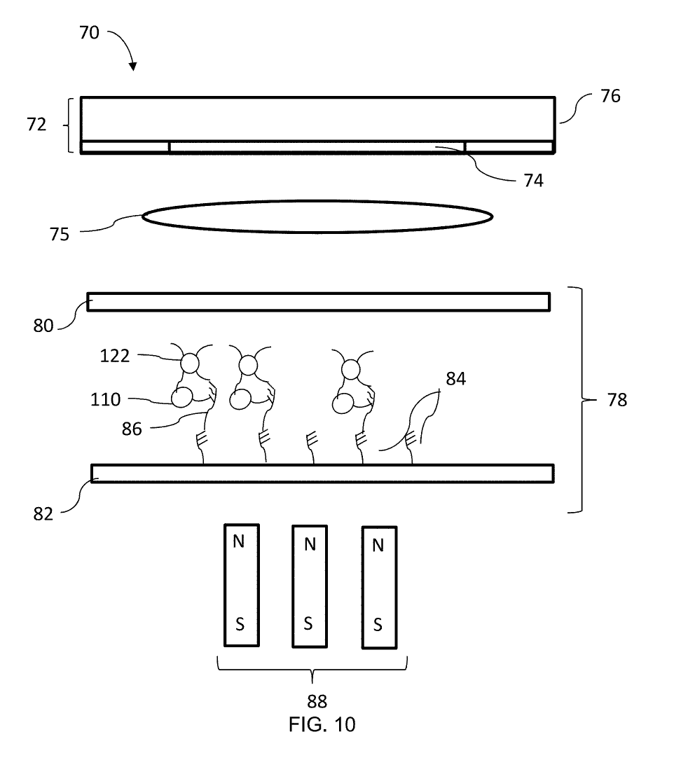

Returning to FIG. 6, at block 98, functionalized nanoparticles 122 are introduced to the flow cell 78 and are bound to the target molecules 86, as illustrated in FIG. 10. In an embodiment, the functionalized nanoparticles 122 are bound directly to the target molecules 86. Alternatively, the functionalized nanoparticles 122 are bound to the magnetic particles 110 bound to each target molecules 86. In an embodiment, a plurality of functionalized nanoparticles 122 are bound to each target molecule 86. Any suitable method of hybridizing or binding the nanoparticles 122 to the target molecules 86 can be used. In an embodiment, the functionalized nanoparticle 122 is a gold particle. In another embodiment, the functionalized nanoparticle 122 is a catalytic nanoparticle, such as a gold catalyst reagent. In an embodiment, the nanoparticles 122 are in the form of catalyst clusters.

In the illustrated embodiment, the nanoparticles 122 are bound to the target molecules 86 after the target molecules 86 are bound to the functionalized surface 82. In an alternative embodiment, the nanoparticles 122 can be bound to the target molecules 86 or magnetic particles 110 prior to binding of the target molecules 86 to the functionalized surface 82.

Following binding of the nanoparticles 122 to the target molecules 86, an optional metallization step can be performed to metallize the nanoparticles 122 and develop or form enlarged nanoparticles or even a film. The developed nanoparticles can improve detection of the target molecules. In this metallization step, the nanoparticles 122 serve as nucleation sites for development of enlarged nanoparticles 124.

Returning to FIG. 6, at block 100, a light source 126 directs a light beam 128 at the target molecules 86 and functionalized nanoparticles 122, 124 in the flow cell 78. The light beam 128 is aimed so that light is directed solely at the target molecules 86 and nanoparticles 122, 124 and no light 128 from the light source 126 is captured by the imager 72. In an embodiment, the flow cell 78 is configured to prevent diffusion of the light beam 128 toward the imager 72.

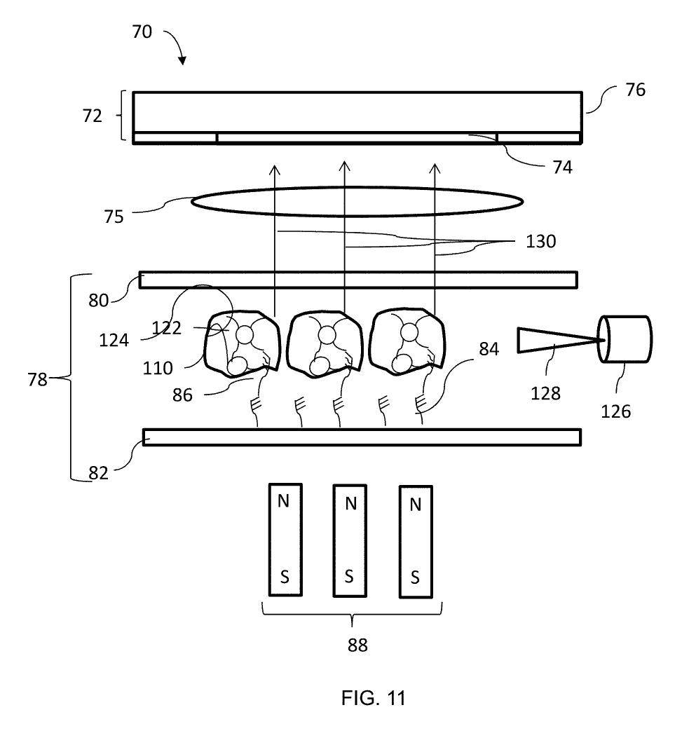

Referring to FIG. 6, at block 102, light 130 (FIG. 11) from the nanoparticles 122, 124, target molecules 86, magnetic particles 110, or a combination thereof, is captured by the imager 72. In an embodiment, the light 130 can be reflected or emitted from the particles 86, 110, 122, 124, or a combination thereof. At block 104, the light 130 captured by the imager 72 is analyzed to detect the presence of target molecules 86. For example, the captured light 130 can be analyzed using dark field microscopy. In this embodiment, the spots of detected light are quantified to determine the presence of target molecules 86.

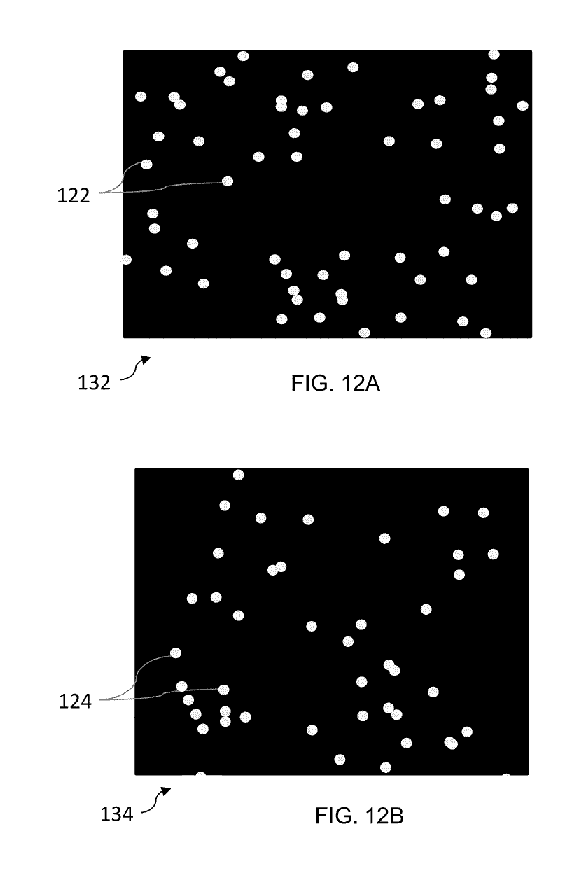

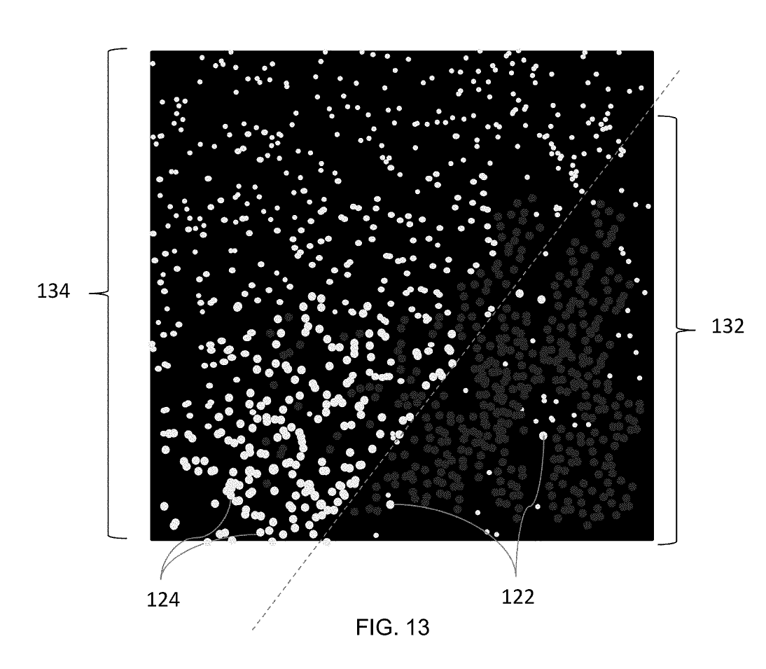

FIGS. 12A-13 illustrate embodiments of light or scattering signatures or captured images of gold nanoparticles 122 captured under dark field microscopy. FIG. 12A is a scattering signature of 50 nm gold nanoparticles and FIG. 12B is a scattering signature of 100 nm gold nanoparticles. FIG. 13 is an image comparing the scattering signature 132 of undeveloped (20 nm) nanoparticles 122 with the scattering signature 134 of developed (100 nm) nanoparticles 124. In an embodiment, a series of dark field images can be captured. In this embodiment, a first image can be captured prior to development of the nanoparticles 122 and at least one additional image can be captured as the nanoparticles are developed. Alternatively, a first image can be captured prior to binding of the target molecules 86 and at least one additional image can be captured following binding of the nanoparticles 122. The captured images can be compared to removed background artifacts and improve analysis of the dark field images.

In an alternative embodiment, a dye particle (not shown) is coupled to the target molecules 86 for detection of the target molecules 86. In this embodiment, the light source 128 is tuned to the wavelength of the dye and regions covered by the dye will fluoresce. The fluoresce is detected by the imager 72.

In another alternative embodiment, to detect the presence of the target molecules 86, following binding of the target molecules 86 and nanoparticles 122, the functionalized surface is exposed to a radiation source (not shown). Upon exposure to the radiation source, the regions of nanoparticles preferentially absorb the radiation, causing localized heating. The localized heating is captured and registered by the imager 72 to detect the presence of the target molecules 86.

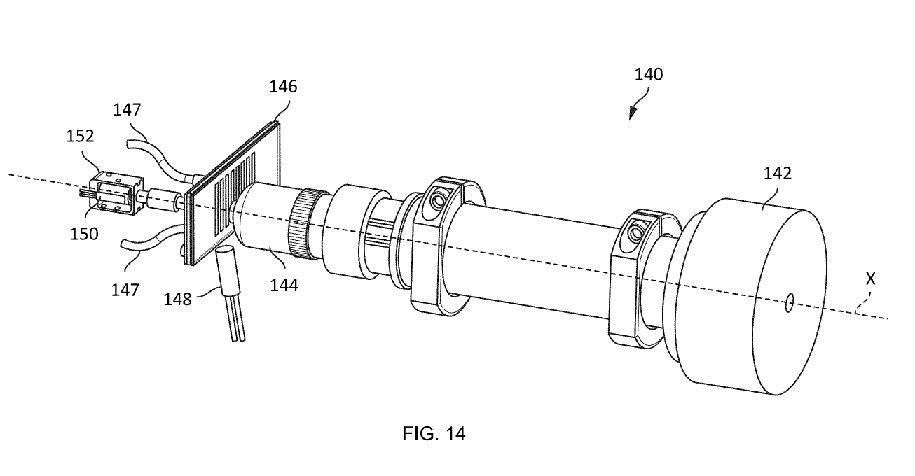

An example of an optical sensor system 140 is illustrated in FIG. 14. Similar to the optical sensor system 70 described above, the optical sensor system 140 includes an imager 142 and an objective or lens 144 coupled to the imager 142. In an embodiment, the imager 142 is a high resolution imager having a wide angle or large field of view. The objective 144 is directed toward the flow cell 146. As discussed above, the interior of the flow cell 146 includes a functionalized surface for binding target molecules. One or more feeder lines 147 can be coupled to the flow cell 146 to facilitate the introduction of various particles to the flow cell 146.

A light source 148 is directed at the flow cell 146. The light source 148 can be any suitable light source. For example, the light source 148 can provide light at a predetermined frequency. For example, the light source 148 can be a white light. The light source 148 is directed or aimed solely at the flow cell 146. In the illustrated embodiment, the light source 148 is directed orthogonally to the axis X on which the objective is positioned. The flow cell 146 is configured to channel the light from the light source 148 toward the particles within the flow cell 146, rather than toward the imager 142 and to prevent light diffusion from the light source 148 to the imager 142.

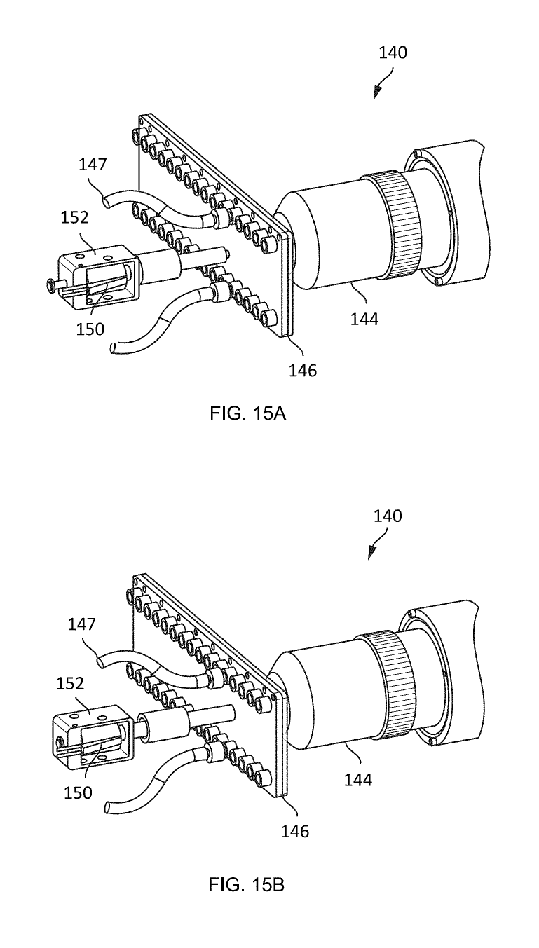

A magnet 150 is positioned opposite the flow cell 146 from the objective 144. An actuator 152, such as a solenoid, is coupled to the magnet 150 and is configured to move the magnet. As illustrated in FIGS. 15A-15B, in an embodiment, the actuator 152 is configured to retract or move the magnet 150 toward (FIG. 15A) the flow cell 146 and to extend or move the magnet 150 away (FIG. 15B) from the flow cell 146.

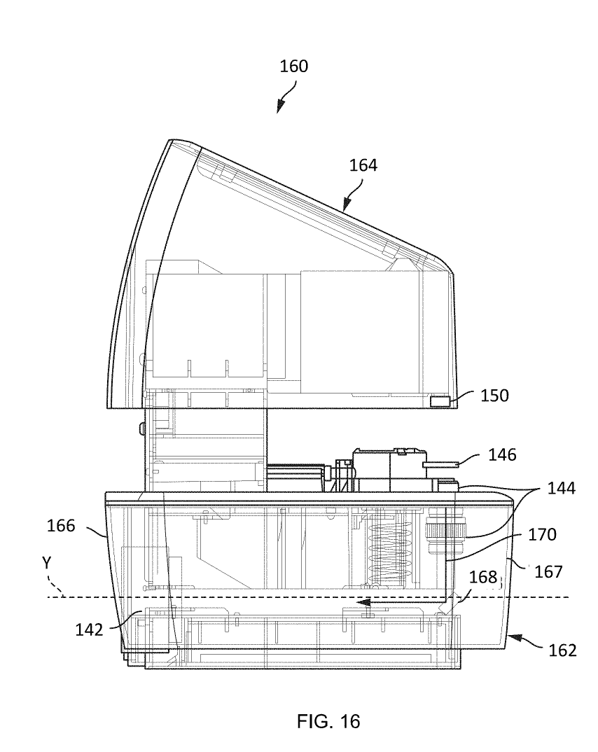

FIG. 16 illustrates an embodiment of an analysis system 160 including an optical system, such as the optical sensor systems 70, 140. In this embodiment, the analysis system 160 includes a base 162 and a head 164. The imager 142 and objective 144 are positioned in the base 162. The flow cell 146 is positioned on the top surface of the base 162, aligned with the objective 144. The magnet 150 is positioned in the head 164 and is configured to extend to and engage with the flow cell 146.

In the illustrated embodiment, in order to minimize the footprint of the analysis system 160, the imager 142 and objective 144 are not aligned along an axis, as illustrated in FIG. 14. Rather, the imager 142 is aligned along an axis Y extending longitudinally through the base 162 between the side surfaces 166, 167 of the base 162. The objective 144 is positioned orthogonal to the axis Y and extends upward through the base 162. A mirror 168 is positioned below the objective 144 and angled toward the imager 142 to create an optical path 170 between the objective 144 and the imager 142.

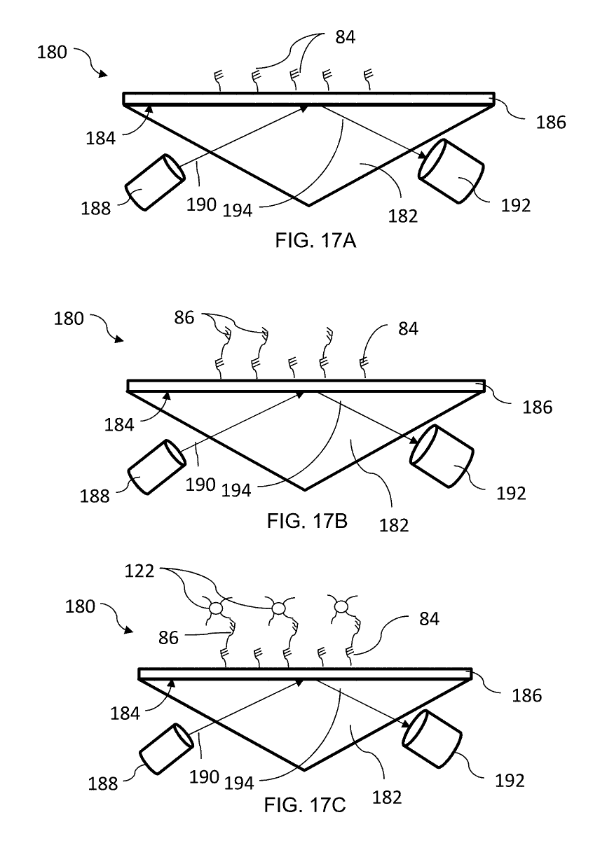

FIGS. 17A-17C illustrate an alternative embodiment of a sensor system 180. In this embodiment, The sensor system 180 includes a prism type substrate 182 having a Kretshmann configuration. The substrate 182 has a surface 184 coated with a metal film 186 suitable for surface plasmon resonance or Raman scattering. For example, the metal film can be gold, silver, copper, titanium, or chromium. The film 186 is functionalized with a bio-specific coating to include capture probes 84. A light source 188 directs a light beam 190 through the prism substrate 182 toward the film 186 and a detector 192 captures light 194 from the film 186, such as light reflected or emitted by the film 186.

In operation, a baseline measurement of the captured light is taken. In an embodiment, the baseline measurement of the captured light is used to calibrate the absorbance angle (FIG. 17A). In addition, the baseline measurement can be sued to identify contaminants or debris on the sensor prior to binding of the target molecules 86 or prior to development of increased nanoparticle size, as discussed below. Following the baseline measurement, a sample containing target molecules 86 is introduced to the sensor system 180 and the target molecules 86 bind to the capture probes 84 (FIG. 17B). In an embodiment, the target molecules 86 can be directed to the surface film 186 via magnetic particles as described above. Functionalized nanoparticles 122 are introduced to the system 180 and allowed to bind to the target molecules 86 (FIG. 17C). A plating bath can optionally be used to increase the size of the bound nanoparticles 122. To detect the presence of the target molecules 86, the beam 190 is directed toward the film 186 and the light 194 from the film 186, such as reflected or emitted, is captured. Any difference in reflectivity or intensity between the baseline measurement and the final measurement is observed in order to detect the presence of the target molecules 86. In an embodiment, the baseline measurement can be used to subtract particles identified as debris from the final measurement.

Possible advantages of the above described method include improved sensitivity of target molecule detection and improved detection of small quantities of target molecules. In addition, the above described method includes increased speed in detection of target molecules. For example, the above described method can permit detection of target molecules without initial replication of the target molecules, such as in a PCR process.

While the present invention has been particularly shown and described with reference to certain exemplary embodiments, it will be understood by one skilled in the art that various changes in detail may be effected therein without departing from the spirit and scope of the invention that can be supported by the written description and drawings. Further, where exemplary embodiments are described with reference to a certain number of elements it will be understood that the exemplary embodiments can be practiced utilizing either less than or more than the certain number of elements.

The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

To the extent that the claims recite the phrase “at least one of” in reference to a plurality of elements, this recitation is intended to mean at least one or more of the listed elements, and is not limited to at least one of each element. For example, “at least one of an element A, element B, and element C,” is intended to indicate element A alone, or element B alone, or element C alone, or any combination thereof. “At least one of element A, element B, and element C” is not intended to be limited to at least one of an element A, at least one of an element B, and at least one of an element C.

### PARTS LIST

A target molecule binding site
B nanoparticle binding site
X axis
Y axis
10 portable assay system
18 rotor
18P port
20 disposable assay cartridge
21 flow cell
22 cartridge body
22B syringe barrel
24 linear actuator
26 plunger shaft
28 elastomeric plunger
30 central chamber
32 assay chamber
34 assay chamber
40 channel
42 channel
44 bottom panel
50 aperture
70 sensor system
72 imager
74 pixel array
75 lens/objective
76 array circuitry
78 flow cell
80 surface
82 functionalized surface
84 capture probes
86 target molecules
88 magnet
90 method
92-104 method steps
110 magnetic particles
110A magnetic particle
110B magnetic particle
112 magnetic core
114 nanoparticle coating
116 magnetic body
118 movement
120 movement
122 nanoparticles
124 enlarged nanoparticles
126 light source
128 light beam
130 light
132 scattering signature (image)
134 scattering signature (image)
140 optical sensor system
142 imager
144 objective/lens
146 flow cell
147 feeder line
148 light source
150 magnet
152 actuator
160 analysis system
162 base
164 head
166 side surface
167 side surface
168 mirror
170 optical path
180 sensor system
182 prism substrate
184 surface
186 film
188 light source
190 light beam
192 detector
194 light

What is claimed is:

1. A system for detecting target molecules in a sample, comprising:
an imager;
a flow cell comprising:
a transparent surface; and
a functionalized surface comprising a plurality of capture probes configured to bind target molecules;
a magnet positioned opposite the functionalized surface, the magnet configured to direct the target molecules to the functionalized surface; and
a light source configured to direct a light beam at bound target molecules,
wherein the imager is configured to capture light from the target molecules to detect the presence of the target molecules.

2. The system of claim 1, wherein a plurality of magnetic particles are configured to bind to the target molecules and wherein the magnet is configured to interact with the magnetic particles to direct the target molecules to the functionalized surface.

3. The system of claim 1, wherein the flow cell is configured to prevent diffusion of the light beam toward the imager.

4. The system of claim 1, wherein the imager is configured to capture dark field images.

5. The system of claim 1, wherein the target molecule is configured to bind a nanoparticle when the target molecule is bound to the functionalized surface and wherein the nanoparticle is configured to reflect the light beam toward the imager.

6. The system of claim 5, wherein the nanoparticle is a gold nanoparticle.

7. The system of claim 5, wherein the nanoparticle is further configured to act as a nucleation site for development of an enlarged nanoparticle.

8. The system of claim 1, further comprising a lens positioned between the imager and the flow cell.

9. A method for detecting a target molecule in a sample with a sensor comprising an imager, a flow cell comprising a functionalized surface having a plurality of capture probes coupled to the functionalized surface, a magnet, and a light source, the method comprising:
binding the target molecule to a magnetic particle;
directing the magnetic particle and target molecule to the functionalized surface via the magnet;
binding the target molecule to one of the plurality of capture probes;
binding a nanoparticle to the target molecule;
directing a light beam from the light source at the nanoparticle;
capturing light from the nanoparticle at the imager; and
analyzing the light from the nanoparticle to detect the target molecule.

10. The method of claim 9, wherein capturing the light comprises capturing a dark field image and wherein analyzing the light comprises quantifying light spots captured in the dark field image.

11. The method of claim 9, further comprising developing an enlarged nanoparticle, wherein the nanoparticle is further configured to act as a nucleation site.

12. The method of claim 9, wherein binding the nanoparticle comprises directly binding the nanoparticle to the target molecule.

13. The method of claim 9, wherein binding the nanoparticle comprises binding the nanoparticle to a binding site of the magnetic particle bound to the target molecule.

14. The method of claim 9, wherein directing the light beam at the nanoparticle comprises preventing diffusion of the light beam toward the imager.

15. The method of claim 9, further comprising denaturing the target molecule to unbind the target molecule from the magnetic particle.

16. The method of claim 9, wherein the magnetic particle comprises a magnetic body and a binding site configured to bind the magnetic particle to the target molecule.

17. The method of claim 16, wherein the magnetic particle further comprises a gold coating.

18. The method of claim 16, wherein the magnetic particle further comprises a nanoparticle binding site.

19. A method for detecting a target molecule in a sample with a sensor comprising an imager, a flow cell comprising a functionalized surface having a plurality of capture probes coupled to the functionalized surface, a magnet, and a light source, the method comprising:
binding the target molecule to a magnetic particle;
directing the magnetic particle and target molecule to the functionalized surface via the magnet;
binding the target molecule to one of the plurality of capture probes;
directing a light beam from the light source at the magnetic particle;
capturing light from the magnetic particle at the imager; and
analyzing the light from the magnetic particle to detect the target molecule.

20. The method of claim 19, wherein the magnetic particle comprises a ferro-gold composite.
